(19) United States
(12) Patent Application Publication
(10) Pub. No.: US 2008/0051459 A1
(43) Pub. Date: Feb. 28, 2008

(54) LONG TERM 24-HOUR INTESTINAL ADMINISTRATION OF LEVODOPA/CARBIDOPA

(75) Inventors: Dag Nyholm, Hannover (DE); Stefan Asberg, Hannover (DE); Roger Bolsoey, Hannover (DE); Mikael Tutschke-Saettler, Hannover (DE)

Correspondence Address: MAYER BROWN LLP, P.O. BOX 2828, CHICAGO, IL, 60690, US

(73) Assignee: Solvay Pharmaceuticals GmbH, Hannover (DE)

(21) Appl. No.: 11/756,297

(22) Filed: May 31, 2007

Related U.S. Application Data
(60) Provisional application No. 60/809,889, filed on May 31, 2006.

(51) Int. Cl. A61K 31/195; A61P 25/16

(52) U.S. Cl. 514/567

(57) ABSTRACT

A method of treating Parkinson's Disease comprising intestinally administering to a patient in need thereof a pharmaceutically effective amount of a composition comprising levodopa and optionally carbidopa continuously over a period of greater than 16 hours.

### RELATED CASES

This application claims priority to U.S. Provisional Application Ser. No. 60/809,889 filed May 31, 2006, which is incorporated herein by reference in its entirety to the extent permitted by law.

### FIELD

The present invention relates to the use of pharmaceutical compositions comprising levodopa and optionally carbidopa for the treatment of Parkinson's Disease (“PD”).

### BACKGROUND

Parkinson's Disease is a progressive disorder; it continues to get worse. For example, as Parkinson's becomes more advanced (“advanced PD”), facial movement, blinking and spontaneous smiling and expression all becomes more difficult, and people have increasing difficulty functioning independently.

Intestinal, e.g., duodenal and/or jejunal administration (via external access point) of a pharmaceutical composition comprising levodopa/carbidopa, such as the composition sold outside the United States under the trade name Duodopa®, has evolved into a treatment alternative in patients with PD. Duodopa is recommended for daytime use only. One reason is that physicians fear the development of tolerance.

Duodopa® (levodopa/carbidopa intestinal gel) may be useful for the treatment of advanced levodopa-responsive PD in which satisfactory control of severe, disabling motor fluctuations and hyper-/dyskinesia cannot be achieved with available combinations of Parkinson medicinal products. Duodopa® is delivered by direct administration (infusion) to the upper small intestine (duodenum or jejunum) by means of the portable, patient controlled CADD-Legacy Duodopa® pump, and requires insertion of a permanent access tube in the abdominal wall, by percutaneous endoscopic gastrostomy (PEG). Prior to insertion of the permanent PEG tube, a positive test of the clinical response to Duodopa® administered via a temporary nasoduodenal tube is recommended for all patients.

Currently, the dose Duodopa® may be administered in three individually adjusted doses: the morning bolus dose, the continuous maintenance dose, and extra bolus doses. The morning bolus dose is administered by the pump to rapidly achieve the therapeutic dose level (e.g., within 10-30 minutes). The total morning dose is usually about 5-10 mL, corresponding to about 100-200 mg levodopa. The total morning dose should not exceed about 15 mL (e.g., about 300 mg levodopa). The maintenance dose is adjustable in steps of about 2 mg/hour (0.1 mL/hour). The continuous maintenance dose should be kept within a range of about 1-10 mL/hour (e.g., about 20-200 mg levodopa/hour) and is usually about 2-6 mL/hour (e.g., about 40-120 mg levodopa/hour). The extra dose should be adjusted individually, normally about 0.5-2.0 mL.

The cassette containing Duodopa® should be attached to the portable pump and the system connected to the nasoduodenal tube or the transabdominal port/duodenal tube for administration just prior to use, according to the instructions provided in the pump instruction manual. The drug cassettes are for single use only and should not be used for longer than one day (up to 16 hours) even if some medicinal product remains. An opened cassette should not be re-used. By the end of the storage time (i.e., after 16 hours in use, or when approaching the expiration date) the gel might become slightly yellow. This does not influence the concentration of the drug or the treatment.

However, because nighttime disability and sleep disturbance are common problems for patients with PD, in particular advanced PD, there is a need for a treatment method that can increase motor performance and improve sleep in patients with PD, in particular advanced PD, without developing clinically relevant tolerance or side effects.

### SUMMARY OF THE DISCLOSURE

In one embodiment, the present disclosure provides pharmaceutical compositions in the form of intestinal gels comprising levodopa and optionally carbidopa for the treatment of PD which are administered continuously over a period of greater than 16 hours per day up to 24 hours per day.

In another embodiment, the present disclosure provides a method of treating PD comprising intestinally (e.g., in the duodenum or jejunum) administering to a patient in need thereof a pharmaceutically effective amount of a composition comprising levodopa and optionally carbidopa continuously over a period of 24 hours.

In another embodiment, the present disclosure provides a method of treating PD comprising intestinally administering to a patient in need thereof a pharmaceutically effective amount of a composition comprising levodopa and optionally carbidopa continuously over a long term period of more than one day.

### BRIEF DESCRIPTION OF THE DRAWINGS

FIG. 1 is a line graph depicting five patients' dose requirements of levodopa/carbidopa over time.

FIG. 2 is a bar graph depicting one patient's PD sleep scale rating over time.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited.

It is to be understood that any ranges, ratios, and ranges of ratios that can be formed by any of the numbers or data present herein represent further embodiments of the present invention. This includes ranges that can be formed that do or do not include a finite upper and/or lower boundary. Accordingly, the skilled person will appreciate that such ratios, ranges, and values are unambiguously derivable from the data presented herein.

As used herein, the term “improve” shall have its plain and ordinary meaning to one skilled in the art of pharmaceutical or medical sciences. Moreover, “improve” shall also mean to ameliorate the effects of PD, or to decrease or lessen a side effect of PD.

As used herein, the term “reduce” or “reducing” shall have its plain and ordinary meaning to one skilled in the art of pharmaceutical or medical sciences. In addition, “reduce” shall mean to diminish or decrease the number of occurrences, the duration, or the intensity, of a PD side effect, such as dyskinesias or hallucinations.

As used herein, the terms “treat” and “treating” shall have their plain and ordinary meaning to one skilled in the art of pharmaceutical or medical sciences. Further, “treat” and “treating” shall mean to improve the quality of life or reduce the symptoms of PD.

As used herein, the terms “dose,” “dose unit,” and/or “dosage unit” refer to a portion of a pharmaceutical composition that contains an amount of a therapeutic agent suitable for a single administration to provide a therapeutic effect. Such dosage units may be administered continuously, one to a small plurality (e.g., 1 to about 4) of times per day, or as many times as needed to elicit a therapeutic response. A particular dosage form can be selected to accommodate any desired frequency of administration to achieve a specified daily dose. Typically one continuous dose unit, one dosage unit, or a small plurality (e.g., up to about 4) of dose units, provides a sufficient amount of the active drug to result in the desired response or effect.

As used herein, the term “therapeutically effective amount” or “therapeutically and/or prophylactically effective amount” refers to an amount of compound or agent that is sufficient to elicit the required or desired therapeutic and/or prophylactic response, as the particular treatment context may require.

It will be understood that a therapeutically and/or prophylactically effective amount of a drug for a patient is dependent inter alia on the body weight of the patient. A “patient” herein to which a therapeutic agent or composition thereof can be administered includes a human subject of either sex and of any age, and also includes any nonhuman animal, particularly a domestic or companion animal, illustratively a cat, dog, or a horse.

Under the present invention, a gel containing levodopa and optional carbidopa is administered via intestinal administration. The gel can be administered (or “infused”) directly into the intestine, e.g., duodenum or the jejunum by a permanent tube via percutaneous endoscopic gastrostomy with an outer transabdominal tube and an inner intestinal tube. In another embodiment, gel can be administered via a radiological gastrojejunostomy. The gel can also be administered via a temporary nasoduodenal tube that is inserted into the patient initially to determine if the patient responds favorably to the treatment method of the present invention before the permanent tube is inserted.

In one embodiment of the present invention, the gel is administered with a portable pump, such as the pump sold under the trade name, CADD-Legacy Duodopa® pump. Specifically, the gel is contained in a cassette, pouch, or vial that is attached to the pump to create the delivery system. The delivery system is then connected to the nasoduodenal tube, the transabdominal port, the duodenal tube, or the jejunum tube for intestinal administration.

In another embodiment, there is disclosed a method of treating PD, comprising intestinally administering to a patient in need thereof a pharmaceutically effective amount of a composition comprising levodopa and, optionally, carbidopa continuously over a period of greater than 16 hours per day.

Additionally, there is disclosed a method of reducing sleep disturbance in a patient with PD, comprising intestinally administering to a patient in need thereof a pharmaceutically effective amount of a composition comprising levodopa and, optionally, carbidopa continuously over a period of greater than 16 hours per day.

In yet another embodiment, there is disclosed a method of improving motor performance in a patient with PD, comprising intestinally administering to a patient in need thereof a pharmaceutically effective amount of a composition comprising levodopa and, optionally, carbidopa continuously over a period of greater than 16 hours per day.

In one embodiment, disclosed is a method of reducing nighttime disabilities in a patient with PD, comprising intestinally administering to a patient in need thereof a pharmaceutically effective amount of a composition comprising levodopa and, optionally, carbidopa continuously over a period of greater than 16 hours per day.

Accordingly, all of the compositions of the present disclosure may be administered continuously over a period of about 17 hours, about 18 hours, about 19 hours, about 20 hours, about 21 hours, about 22 hours, about 23 hours or about 24 hours. Further, the compositions may be administered continuously over a period of about 26 hours, about 28 hours, about 30 hours, about 32 hours, about 34 hours, about 36 hours, about 38 hours, about 40 hours, about 42 hours, about 44 hours, about 46 hours, about 48 hours, or longer.

In addition, the present disclosure relates to treating patients utilizing a composition comprising levodopa and, optionally, carbidopa in the form of particles suspended in an aqueous carrier, the particles having a maximum particle size not exceeding about 80 μm and that said carrier has a viscosity of at least 300 mPas, at a moderate shear rate. In one embodiment, the particles are micronized. Micronization and particle size distribution analysis are performed by Micron Technologies UK. The particles may be characterized by a D90 value of about 20 Mm or less. Such particles may also be characterized by a D50 value of about 5 μm or less. In another embodiment, the maximum particle size does not exceed about 70 μm, about 60 μm, about 50 μm, about 40 μm, or about 30 μm. In yet another embodiment, the carrier has a viscosity of about 350 mPas, about 400 mPas, about 450 mPas, about 500 mPas, about 550 mPas, or about 600 mPas, at a moderate shear rate.

Such composition may be formulated such that the weight ratio of levodopa and carbidopa ranges from about 10:1 to about 1:1, or from about 5:1 to about 2:1, or from about 4.5:1 to about 3.5:1, or wherein the weight ratio is about 4:1.

In another embodiment, the dose of the levodopa and/or carbidopa gel is adjusted to optimize the clinical response achieved by a patient, which means maximizing the functional ON-time during the day by minimizing the number and duration of OFF-time episodes (i.e., bradykinesia) and minimizing ON-time with disabling dyskinesia.

In yet another embodiment, PD patients who are given levodopa and optionally carbidopa gel continuously over 24 hours experience an increase in sleep quality.

Under one embodiment of the present invention, the levodopa and optionally carbidopa gel is given as a monotherapy. In another embodiment, the levodopa and/or carbidopa gel is given concurrently with other medicinal products used in the treatment of PD.

In one embodiment, the dose of levodopa received by a patient according to methods of the present invention may be, for example, about 20 to about 5000 mg, about 20 mg to about 4000 mg, about 20 mg to about 3000 mg, about 20 mg to about 2000 mg, or about 20 mg to about 1000 mg per day. For example, a patient according to methods of the present invention may receive about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg of levodopa per day.

In another embodiment, the dose of carbidopa received by a patient according to methods of the present invention may be, for example, 0 to about 625 mg, 0 mg to about 500 mg, 0 mg to about 375 mg, 0 mg to about 250 mg, or 0 mg to about 125 mg per day. For example, a patient according to methods of the present invention may receive about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, or 1250 mg of carbidopa per day.

In another embodiment, the present invention comprises a pharmaceutical composition that is a gel comprising levodopa and carbidopa in a ratio of 4 to 1. In one embodiment, the formulation (Duodopa®) comprises the following ingredients (w/w):

Levodopa 2.0%

Carbidopa monohydrate 0.5%

Carmellose sodium 2.92%

qs to 100% with purified water

In another embodiment, continuous intestinal administration of a pharmaceutical composition comprising levodopa and optionally carbidopa reduces the motor fluctuations and increases the “on”-time for patients with advanced PD who have previously received tablet treatment with levodopa/decarboxylase inhibitor. The motor fluctuations and hyper-/dyskinesias are reduced by the present invention due to the fact that the plasma concentrations of levodopa are kept at a steady level within the individual therapeutic window. In yet another embodiment, therapeutic effects on motor fluctuations and hyper-/dyskinesias are achieved during the first treatment day.

Applicant has unexpectedly found that low tolerance or low tachyphylaxis results from continuous intestinal administration of the levodopa/carbidopa composition described herein. Moreover, in one embodiment, the dose of the continuous intestinal administration of the levodopa/carbidopa was increased over a period of one year, yet no increase in side-effects such as dyskinesias or hallucinations was observed. In yet another embodiment, the dose of the continuous intestinal administration of the levodopa/carbidopa was increased over a period of two years, yet no increase in side-effects such as dyskinesias or hallucinations was observed. In still another embodiment, the dose of the continuous intestinal administration of the levodopa/carbidopa was increased over a period of three years, yet no increase in side-effects such as dyskinesias or hallucinations was observed.

In addition, the half-life of levodopa is lengthened with continuous administration.

Accordingly, the present compositions can be continuously administered intestinally without the need to orally administer levodopa/carbidopa during the night to aid sleep. As shown in the example below, sleep is improved and other disabilities associated with PD are reduced when the composition is administered continuously. In one embodiment, patients experienced improved sleep quality with the intestinal administration of levodopa and carbidopa. In yet another embodiment, patients who were examined with PDSS reported an increase in total score by 130% (from 53 to 122) from one night to another, when around-the-clock administration was initiated. In still another embodiment, patients who were examined with PDSS reported an increase in total score by about 100%, 110%, 120%, 130%, 140%, 150%, 160%, 170%, 180%, 190%, or 200% from one night to another, when around-the-clock administration was initiated. In yet another embodiment, the improvement in PDSS score was shown to be persistent at a follow-up two years later.

In one embodiment, the present invention encompasses the use of a composition comprising 20 mg/mL of levodopa, 5 mg/mL of carbidopa, a thickening agent (e.g., cellulose), and water.

Compositions of the invention optionally comprise one or more additional pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition.

Illustrative excipients include antioxidants, agents to adjust the pH and osmolarity, preservatives, thickening agents, colorants, buffering agents, bacteriostats, and stabilizers. Generally speaking, a given excipient, if present, will be present in an amount of about 0.001% to about 95%, about 0.01% to about 80%, about 0.02% to about 25%, or about 0.3% to about 10%, by weight.

Illustrative antioxidants for use in the present invention include, but are not limited to, butylated hydroxytoluene, butylated hydroxyanisole, potassium metabisulfite, and the like.

Illustrative agents that increase viscosity (e.g., thickening agents) include, but are not limited to, cellulose, methylcellulose, carboxymethylcellulose sodium, ethylcellulose, carrageenan, carbopol, and/or combinations thereof.

In one embodiment, compositions of the invention optionally comprise a buffering agent. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, or a magnesium buffering agent. Suitable buffering agents include carbonates, phosphates, bicarbonates, citrates, borates, acetates, phthalates, tartrates, succinates of any of the foregoing, for example sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). Furthermore, combinations or mixtures of any two or more of the above mentioned buffering agents can be used in the pharmaceutical compositions described herein.

The foregoing excipients can have multiple roles as is known in the art. Therefore, classification of excipients above is not to be construed as limiting in any manner.

These and many other aspects of the invention will be fully apparent to one of ordinary skill in the art in view of the example set forth below. The example provided herein is illustrative and is not to be construed as limiting the invention in any manner.

### EXAMPLE 1

Most patients with PD suffer from sleep disturbance. In advanced PD, dopaminergic medication is sometimes frequently used during nighttime to improve sleep. Continuous 24-hour administration of levodopa has only been investigated in a very small number of patients and during short periods of time because of the fear of tolerance development and psychiatric side-effects. Five cases of 24-hour duodenal administration of levodopa/carbidopa (Duodopa®) for up to 37 months were studied.

Method

The hospital charts were reviewed retrospectively in five PD patients who were given continuous 24-hour duodenal administration of levodopa/carbidopa in a 4:1 ratio by weight in a gel via intestinal administration (Duodopa®). The formulation is described in U.S. Pat. No. 5,635,213, which is incorporated herein by reference. Dosage, efficacy, sleep pattern, and side-effects were recorded.

The patients were treated with this regime for their well-being. Thus, no prospective study protocols were used. PD Sleep Scale (“PDSS”) was used in one case to assess the impact of the administration on sleep.

Demographic and clinical data are shown below in Table One. All patients had “on-off” fluctuations on individually optimized combinations of conventional drugs and all experienced nocturnal akinesia. Duodenal levodopa administration, initially daytime only, led to a marked reduction of motor fluctuations and dyskinesias in all patients. On 24-hour administration all patients were initially given lower administration rates at night, but three eventually needed the optimal dose around-the-clock. Daily mean dosage at latest follow-up was 3,015 mg levodopa (range 2,002 mg to 4,320 mg).

TABLE ONE
Patient Demographics Dosage & Outcome

| | Case No. | | | | | |
|---|---|---|---|---|---|---|
| | 1 | 2 | 3 | 4 | 5 | Mean |
| Sex | M | M | M | M | M | |
| Age at onset of PD | 35 | 37 | 35 | 43 | 47 | 39 |
| Symptoms (Years) | | | | | | |
| Age at initiation of oral | 36 | 40 | 38 | 44 | 52 | 42 |
| levodopa therapy (years) | | | | | | |
| Age at initiation of 24-hour | 52 | 47 | 61 | 52 | 64 | 55 |
| administration therapy | | | | | | |
| (years) | | | | | | |
| Medication prior to | br, en, | br, en, | ap-inf | am, ap-inf | ap-inf | |
| administration, concomitant | se | ro | (24) | (16), en, ro | (16), ap- | |
| to levodopa/decarboxylase | | | | | inj, br, ca, | |
| inhibitor | | | | | en, ro, to | |
| Duration of 24-hour | 20 | 29 | 37 | 16 | 13 | 23 |
| administration (months) | | | | | | |
| Daily levodopa dose at | 2002 | 2544 | 4320 | 3408 | 2800 | 3015 |
| latest follow-up (mg) | | | | | | |
| Change in daytime | −2 | 29 | 38 | 12 | −6 | 14 |
| administration | | | | | | |
| rate during 24-hour | | | | | | |
| administration from | | | | | | |
| initiation up to latest follow- | | | | | | |
| up (%) | | | | | | |
| Motor performance at latest | Stable | Stable | Stable | Fluctuating | Stable | |
| follow-up | | | | | | |

PD = Parkinson's Disease;
br = bromocriptine;
en = entacapone;
se: selegiline;
ro = ropinirole;
ap-inf = apomorphine administration (16-hour or 24-hour);
am = amantadine;
ap-inj = apomorphine injection;
ca = cabergoline;
to = tolcapone

Case 1 was initially treated with daytime administration for 2.5 years. On 24-hour administration, sleep was markedly improved and the patient was able to sleep for 6 hours, which he had not been able to do for many years. Self-scoring on the PDSS increased from 53 to 122 the morning after his first night on continuous administration. At latest follow-up, both motor function and sleep were good.

Case 2 had earlier experienced side effects including hallucinations on dopamine agonists. After 1 year of daytime administration, continuous 24-hour administration substantially improved both motor function and sleep at night. Motor performance was stable at latest follow-up.

Case 3 was treated with levodopa/carbidopa hourly and 24-hour administration of apomorphine the preceding 4 years. Mild nighttime hallucinations had been treated with clozapine for several years. Although the levodopa dose was increased to extremely high levels, clozapine could be discontinued without any impairment in the hallucinations. After 3 years of around-the-clock levodopa administration, now at 180 mg/hour, the patient had essentially no motor fluctuations at all.

Case 4 began around-the-clock administration after 1 week of daytime administration. Daytime motor performance was stable but he still had dystonia and fragmented sleep pattern at nights. For 3 months amantadine was added due to hyperkinesia, and his motor performance was improved. The patient now frequently changes his administration rate thus mimicking oral therapy. His “on-off” fluctuations were in the same magnitude as with oral combination therapy at latest follow-up.

Case 5 started around-the-clock administration 2 weeks after initiation of administration therapy. His motor performance and especially his sleep pattern improved on this regime. He has had one single episode of hallucinations that occurred after a period of sleep deprivation and undernourishment. His motor performance has remained stable with only mild dystonia in a leg.

Results

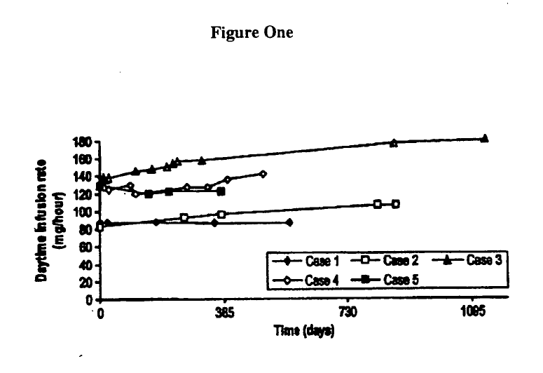

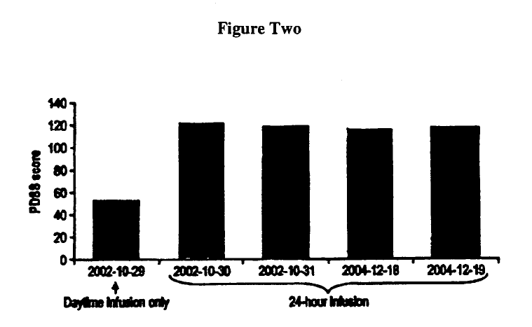

At the latest observation the patients had been treated with continuous administration for 23 months (range 13-37 months). All had relatively high doses of levodopa (monotherapy). Dose requirement increased in three patients over time, but the stable response to duodenal levodopa administration was maintained according to clinical observations in all but one. (See FIG. 1). Initially, all patients were given lower administration rates at nights, but eventually three needed the optimal dose around-the-clock. No increase in side-effects such as dyskinesias or hallucinations was observed. All patients experienced improved sleep quality with administration. The patient who was examined with PDSS reported an increase in total score by 130% (from 53 to 122) from one night to another, when around-the-clock administration was initiated, see FIG. 2. The improvement in PDSS score was shown to be persistent at a follow-up two years later.

Discussion

The 24-hour duodenal levodopa administration therapy has, for our five patients, replaced frequent oral drug intakes at night. The mean change in administration rate was +14% over a mean treatment period of about 2 years. Previous long-term experience from around-the-clock levodopa administration is limited to one patient, where the administration rate had to be increased from 86 to 100 mg/hour (16%) in about 5 weeks. Such a rapid increase in dose requirement was not seen in any of our patients. The dosage was decreased with time in two patients. No increase in dyskinesias or hallucinations was observed. The stable response to levodopa administration was maintained in all patients but one. Thus, continuous 24-hour duodenal levodopa administration can increase motor performance and improve sleep in patients with advanced PD without developing clinically relevant tolerance or side effects.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

What is claimed:

1. A method of treating Parkinson's Disease, comprising intestinally administering to a patient in need thereof a pharmaceutically effective amount of a composition comprising levodopa and, optionally, carbidopa continuously over a period of greater than 16 hours per day.

2. The method of claim 1 wherein said patient has advanced Parkinson's Disease.

3. The method of claim 1 or 2 wherein the composition is administered up to 24 hours per day.

4. The method of claim 3 wherein the composition is administered continuously over a period of 24 hours per day.

5. The method of claim 1 wherein the administration is continued as a long-term treatment for more than 1 day.

6. The method of claim 4 wherein the administration is continued as a long-term treatment for more than 1 day.

7. A method of reducing sleep disturbance in a patient with Parkinson's Disease, comprising intestinally administering to a patient in need thereof a pharmaceutically effective amount of a composition comprising levodopa and, optionally, carbidopa continuously over a period of greater than 16 hours per day.

8. The method of claim 7 wherein said patient has advanced Parkinson's Disease.

9. The method of claim 7 or 8 wherein the composition is administered up to 24 hours per day.

10. The method of claim 9 wherein the composition is administered continuously over a period of 24 hours per day.

11. The method of claim 7 wherein the administration is continued as a long-term treatment for more than 1 day.

12. The method of claim 11 wherein the administration is continued as a long-term treatment for more than 1 day.

13. A method of improving motor performance in a patient with Parkinson's Disease, comprising intestinally administering to a patient in need thereof a pharmaceutically effective amount of a composition comprising levodopa and, optionally, carbidopa continuously over a period of greater than 16 hours per day.

14. The method of claim 13 wherein said patient has advanced Parkinson's Disease.

15. The method of claim 13 or 14 wherein the composition is administered up to 24 hours per day.

16. The method of claim 15 wherein the composition is administered continuously over a period of 24 hours per day.

17. The method of claim 13 wherein the administration is continued as a long-term treatment for more than 1 day.

18. The method of claim 16 wherein the administration is continued as a long-term treatment for more than 1 day.

19. A method of reducing nighttime disabilities in a patient with Parkinson's Disease, comprising intestinally administering to a patient in need thereof a pharmaceutically effective amount of a composition comprising levodopa and, optionally, carbidopa continuously over a period of substantially greater than 16 hours per day.

20. The method of claim 19 wherein said patient has advanced Parkinson's Disease.

21. The method of claim 19 or 20 wherein the composition is administered up to 24 hours per day.

22. The method of claim 21 wherein the composition is administered continuously over a period of 24 hours per day.

23. The method of claim 19 wherein the administration is continued as a long-term treatment for more than 1 day.

24. The method of claim 22 wherein the administration is continued as a long-term treatment for more than 1 day.

25. The method of any of claims 1, 7, 13 and 19 wherein the composition comprising levodopa and, optionally, carbidopa in form of particles suspended in an aqueous carrier, said particles having a maximum particle size not exceeding 80 μm and that said carrier has a viscosity of at least 300 mPas, at a moderate shear rate.

26. The method of claim 25 wherein the particles are characterized by a D90 value of about 20 μm or less.

27. The method of claim 26 wherein the particles are characterized by a D50 value of about 5 μm or less.

28. The method of any of claims 1, 7, 13, and 19 wherein the daily dose of levodopa ranges from about 0.5 mg to about 5000 mg.

29. The method of claim 28 wherein the daily dose of levodopa ranges from about 20 mg to about 4000 mg.

30. The method of claim 28 wherein the daily dose of levodopa ranges from about 20 mg to about 3000 mg.

31. The method of claim 28 wherein the daily dose of levodopa ranges from about 20 mg to about 2000 mg.

32. The method of claim 28 wherein the daily dose of levodopa ranges from about 20 mg to about 1000 mg.

33. The method of any of claims 1, 7, 13, and 19 wherein the weight ratio of levodopa and carbidopa ranges from about 10:1 to about 1:1.

34. The method of claim 33 wherein the weight ratio ranges from about 5:1 to about 2:1.

35. The method of claim 33 wherein the weight ratio ranges from about 4.5:1 to about 3.5:1.

36. The method of claim 33 wherein the weight ratio is about 4:1.

37. The method of any of claims 1, 7, 13, and 19 wherein said composition is in form of a gel.
